(19) United States
(12) Patent Application Publication
(10) Pub. No.: US 2024/0335358 A1
(43) Pub. Date: Oct. 10, 2024

(54) CONTAINER LOCKING LID SYSTEM

(71) Applicants: William Ryan Tanner, Farmington, UT (US); Caleb Gerald Greaves, Salt Lake City, UT (US); Barry Keith Hanover, Park City, UT (US)

(72) Inventors: William Ryan Tanner, Farmington, UT (US); Caleb Gerald Greaves, Salt Lake City, UT (US); Barry Keith Hanover, Park City, UT (US)

(21) Appl. No.: 18/132,923

(22) Filed: Apr. 10, 2023

(51) Int. Cl. A61J 7/04; A61J 7/00; B65D 51/24; B65D 55/02; B65D 83/04; G16H 20/13

(52) U.S. Cl. CPC A61J 7/0436; A61J 7/0076; B65D 51/245; B65D 55/02; B65D 83/0409; G16H 20/13; A61J 2200/74; B65D 2583/0404; B65D 2583/0477

(57) ABSTRACT

A system includes a container forming an interior volume configured to store products and a lid assembly configured to secure to the container. The lid assembly includes a lid housing forming an opening. The products are to be dispensed from the interior volume via the opening. The lid assembly further includes a dispensing structure configured to load a portion of the products to be dispensed. The lid assembly further includes a cover structure configured to selectively unblock the opening to allow the portion of the products to be dispensed. The lid assembly further includes a processing device configured to determine that the portion of the products is to be dispensed and cause the cover structure to be actuated to unblock the opening to allow dispensing of the portion of the products.

### TECHNICAL FIELD

The present disclosure relates to bottle locking lids, more particularly, container locking lids.

### BACKGROUND

Medications are used to diagnose, cure, treat, and/or prevent medical conditions. Prescription medication is provided for use by a specific user that has a prescription from a medical professional for that specific type of prescription medication.

### SUMMARY

The following is a simplified summary of the disclosure in order to provide a basic understanding of some aspects of the disclosure. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In one aspect of the disclosure a system includes a container forming an interior volume configured to store products and a lid assembly configured to secure to the container. The lid assembly includes a lid housing forming an opening, wherein the products are to be dispensed from the interior volume via the opening. The lid assembly further includes a dispensing structure configured to load a portion of the products to be dispensed and a cover structure configured to selectively unblock the opening to allow the portion of the products to be dispensed. The lid assembly further includes a processing device configured to determine that the portion of the products is to be dispensed and to cause the cover structure to be actuated to unblock the opening to allow dispensing of the portion of the plurality of products.

In another aspect of the disclosure a locking lid assembly includes a lid housing forming an opening, where the housing is configured to secure a container. The container forms an interior volume configured to store products. The products are to be dispensed from the interior volume via the opening. The locking lid assembly further includes a dispensing structure configured to load a portion of the products to be dispensed and a cover structure configured to selectively unblock the opening to allow the portion of the plurality of products to be dispensed. The locking lid assembly further includes a processing device configured to determine that the portion of the products is to be dispensed and to cause the cover structure to be actuated to unblock the opening to allow dispensing of the portion of the products.

In another aspect of the disclosure a locking lid assembly includes a lid housing forming an interior volume, the lid housing further forming a lid housing opening coupled to the interior volume. The locking lid assembly further includes a substantially cylindrical dispensing structure disposed in the interior volume of the lid housing, the substantially cylindrical dispensing structure forming a dispensing structure opening configured to receive one or more products. The substantially cylindrical dispensing structure is configured to rotate to cause the one or more products disposed in the dispensing structure opening to be dispensed through the lid housing opening.

### BRIEF DESCRIPTION OF THE DRAWINGS

The present disclosure is illustrated by way of example, and not by way of limitation, in the figures of the accompanying drawings in which like references and/or reference numbers indicate similar elements. It should be noted that different references to “an,” “one,” or “some” embodiment(s) in this disclosure are not necessarily to the same embodiment or embodiments, and such references mean at least one.

FIGS. 1A-B illustrate views of container locking lid systems, according to certain embodiments.

FIG. 2 illustrates a perspective top view of a container locking lid system, according to certain embodiments.

FIG. 3 illustrates an exploded perspective top view of a container locking lid system, according to certain embodiments.

FIGS. 4A-B illustrate a user interface of a container locking lid system, according to certain embodiments.

FIGS. 5A-B illustrate a cover structure of a container locking lid, according to certain embodiments.

FIGS. 6A-B illustrate a container locking lid system, according to certain embodiments.

FIGS. 7A-B illustrate a dispenser of a container locking lid system, according to certain embodiments.

FIG. 8 is a block diagram illustrating a computer system, according to certain embodiments.

### DETAILED DESCRIPTION

Described herein are technologies related to container locking lid systems (e.g., prescription bottle locking lid systems).

Medications are used to diagnose, cure, treat, and/or prevent medical conditions. Prescription medication is provided for use by a specific user that has a prescription from a medical professional for that specific type of prescription medication. In the United States, it is estimated that 4.55 billion prescriptions were filled during the year 2020. Currently, an estimated 2.7 million individuals in the United States suffer some kind of substance use disorder related to opioid pharmaceuticals. In 2021, over 75,000 people died from opioid overdoses in the United States. This crisis has impacted individuals across all demographics, including those with no prior history of substance abuse. The economic burden of the crisis is also substantial, with increased healthcare costs, lost productivity, and the need for addiction treatment services. The development of effective interventions and treatments to address the opioid epidemic is therefore of critical importance.

Conventionally, when a medication (e.g., pill) prescription is filled at a pharmacy, the prescription medication is provided to a user in an allegedly ‘child-proof’ bottle housing the pills, with instructions on how and when to take the medication. However, for many American adults, receiving a large amount of opioids in a single instance can prove too much of a temptation to misuse the medication. Conventional prescription medication containers do not help solve the problem or mitigate the risk inherent in opioid prescription medication use. Addressing this crisis requires innovative solutions to prevent the abuse of prescription opioids.

Existing Conventional solutions fail to meet this need. Some conventional solutions use timers to remind patients about adherence to the prescription schedule, but do not stop users from taking more than their prescribed dosage at prescribed intervals. Conventional locking caps are to be squeezed to be removed can prevent young children from accessing the medication, but the actual user to whom the medication is prescribed still has access to all the pills at once.

As such, there is a need for a solution that (i) provides prescribed medication (e.g., pills) to those who need the prescribed medication (ii) in a safe, controlled manner that mitigates the temptation to misuse the prescribed medication.

The systems, devices, and methods of the present disclosure provide a container locking lid system that provides solutions to shortcomings of conventional systems.

In some embodiments, a system (e.g., container locking lid system, prescription bottle locking lid system) includes a container (e.g., medication bottle, prescription bottle, etc.) forming an interior volume configured to store products (e.g., medication in solid dosage form, pills, tablets, etc.) and a lid assembly configured to secure to the container. In some embodiments, the lid assembly may include a lid housing forming an opening. In some embodiments, the products are to be dispensed from the interior volume via the opening. In some embodiments, the lid assembly may include a dispensing structure configured to load a portion of the products to be dispensed. In some embodiments, the lid assembly may include a cover structure configured to selectively unblock the opening to allow the portion of the products to be dispensed. In some embodiments, the lid assembly may include a processing device configured to determine that the portion of the products is to be dispensed and to cause the cover structure to be actuated to unblock the opening to allow dispensing of the portion of the products.

In some embodiments, a locking lid assembly includes a lid housing forming an opening, where the housing may be configured to secure a container, the container forming an interior volume configured to store products. In some embodiments, the products may be dispensed from the interior volume via the opening. In some embodiments, the locking lid assembly may include a dispensing structure configured to load a portion of the products to be dispensed. In some embodiments, the locking lid assembly may include a cover structure configured to selectively unblock the opening to allow the portion of the products to be dispensed. In some embodiments, the locking lid assembly may include a processing device configured to determine that the portion of the plurality of products is to be dispensed and cause the cover structure to be actuated to unblock the opening to allow dispensing of the portion of the products.

In some embodiments, a locking lid assembly may include a lid housing forming an interior volume, the lid housing further forming a lid housing opening coupled to the interior volume. In some embodiments, the locking lid assembly may include a substantially cylindrical dispensing structure disposed in the interior volume of the lid housing, the substantially cylindrical dispensing structure forming a dispensing structure opening configured to receive one or more products, where the substantially cylindrical dispensing structure is configured to rotate to cause the one or more products disposed in the dispensing structure opening to be dispensed through the lid housing opening.

The systems, devices, and methods of the present disclosure have advantages over conventional solutions. The present disclosure stops users from taking more than the prescribed dosage at prescribed intervals, does not allow access to all the pills at once, and prevents any demographic (e.g., young children and adults) from accessing medication outside of prescribed intervals and dosages.

Although the present disclosure describes use of a container locking lid system 100 with prescription medication (e.g., pills, etc.), in some embodiments, the container locking lid system can be used with liquids, solids, vitamins, over-the-counter medication, etc.

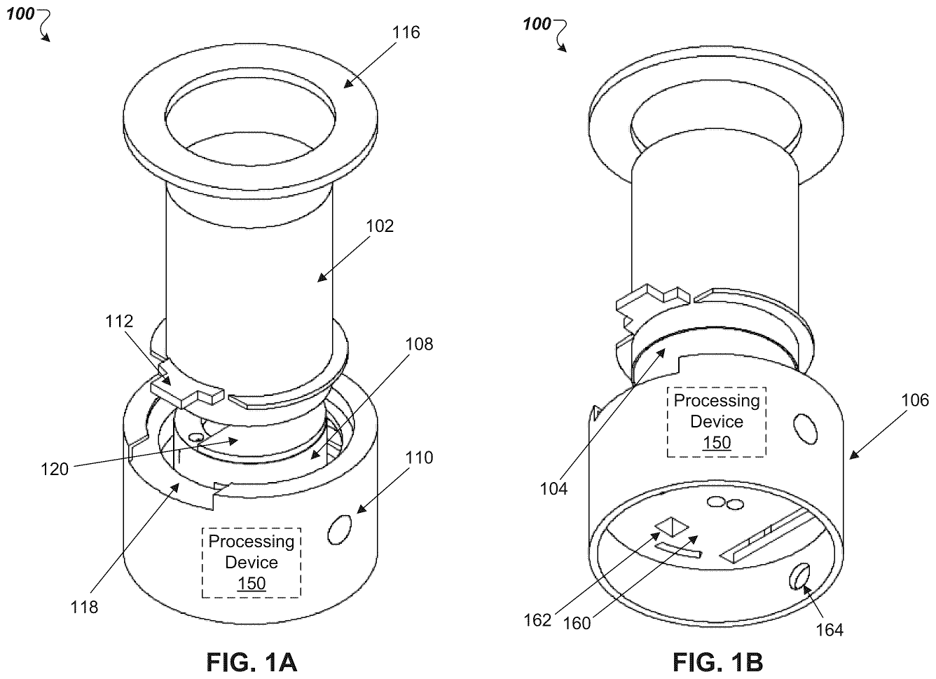

FIGS. 1A-B illustrate views of a container locking lid system 100, according to certain embodiments. FIG. 1A illustrates a top perspective view and FIG. 1B illustrates a bottom perspective view. In some embodiments, container locking lid system 100 of FIGS. 1A-B are the same container locking lid system 100.

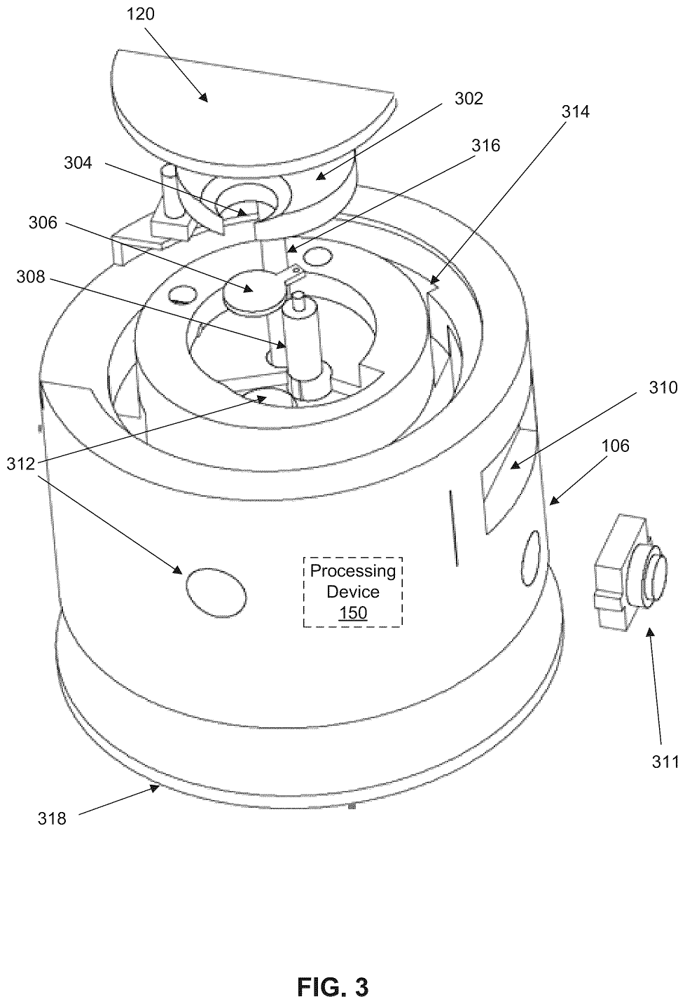

In some embodiments, the container locking lid system 100 may include a container 102 and a lid assembly 106. The container 102 (e.g., prescription bottle) may form an interior volume 104 configured to store products (e.g., medication, medication in solid dosage form, pills, tablets, etc.). The lid assembly 106 (e.g., locking lid assembly) may be configured to secure to container 102. In some embodiments, lid assembly 106 may include a lid housing 108 forming an opening 110, where the products are to be dispensed from interior volume 104 via opening 110 of lid housing 108. In some embodiments, the lid assembly 106 may include a dispensing structure (not shown in FIG. 1, e.g., dispensing structure 302 of FIG. 3) configured to load a portion of the plurality of products to be dispensed. In some embodiments, lid assembly 106 may include a cover structure (not shown in FIG. 1, e.g., cover structure 306 of FIG. 3) configured to selectively unblock opening 110 to allow the portion of the products to be dispensed. In some embodiments, the lid assembly 106 may include a processing device 150 configured to determine that the portion of the plurality of products is to be dispensed and to cause the cover structure (e.g., cover structure 306 of FIG. 3) to be actuated to unblock opening 110 to allow dispensing of the portion of the plurality of products.

In some embodiments, container 102 may be a medication bottle. In some embodiments, the container may be any type of container holding a product to be dispensed. In some embodiments, container 102 may be a prescription bottles. In some embodiments, a prescription bottle (e.g., prescription pill bottle) may come in various sizes and dimensions. In some embodiments, prescription bottle sizes and dimensions vary based on the amount and size of medication (e.g., pills) they contain.

In some embodiments, container 102 may be a 13 dram prescription bottle (e.g., about 2.15 inches to 2.25 inches in height and about 1.0 inch to 1.2 inches in diameter). In some embodiments, a 13 dram bottle may be used for small prescriptions or short-term medications.

In some embodiments, container 102 may be a 16 dram prescription bottle (e.g., about 2.25 inches to 2.4 inches in height and about 1.2 inches to 1.3 inches in diameter). In some embodiments, a 16 dram bottle may be slightly larger than the 13 dram bottle.

In some embodiments, container 102 may be a 20 dram prescription bottle (e.g., about 2.4 inches to 2.6 inches in height and about 1.3 inches to 1.4 inches in diameter). In some embodiments, a 20 dram bottle may be used for medium-term medication prescriptions.

In some embodiments, container 102 may be a 30 dram prescription bottle (e.g., about 2.75 inches to 3.1 inches in height and about 1.4 inches to 1.5 inches in diameter). In some embodiments, a 30 dram bottle may be used for long-term medications.

In some embodiments, container 102 may be a 40 dram prescription bottle (e.g., about 3.1 inches to 3.8 inches in height and about 1.5 inches to 1.7 inches in diameter) In some embodiments, a 40 dram bottle may be a large bottle that is used for medications that come in large quantities.

In some embodiments, container 102 may be a 60 dram prescription bottle (e.g., about 3.8 inches to 4.5 inches in height and about 1.9 inches to 2.1 inches in diameter In some embodiments, a 60 dram bottle may be the largest size of prescription pill bottle and may be used for medications that come in large quantities or need to be stored for a long period of time.

In some embodiments, the actual dimensions of container 102 (e.g., prescription pill bottle) may vary depending on the manufacturer or the specific design of the bottle. In some embodiments, the color of container 102 (e.g., prescription bottle) and labeling may also vary (e.g., depending on the pharmacy or prescription requirements).

In some embodiments, container 102 may be a prescription bottle that has a locking tab 112 (e.g., container forms a protrusion) that causes container 102 to be secured to a lid assembly 106, according to certain embodiments. In some embodiments, locking tab 112 of the prescription bottle may be (i) undepressed (e.g., at a resting position, no external force applied to the locking tab) or (ii) depressed (e.g., external force applied to locking tab 112, locking tab 112 displaced from the undepressed position).

In some embodiments, container 102 may be a prescription pill bottle. In some embodiments, container 102 may have a tab to be pressed in order for the lid to rotate (e.g., lid assembly 106). In some embodiments, the lid assembly 106 is locked onto container 102 by way of the tab. Lid assembly 106, may be rigid, preventing the tab from being depressed and preventing the lid assembly 106 from being able to be rotated off of container 102.

The container 102 (e.g., prescription bottle) that has a locking tab 112 secures to lid assembly 106, according to certain embodiments. In some embodiments, locking tab 112 of the prescription bottle is first placed into a housing 108 of the lid assembly 106. In response to locking tab 112 being placed into housing 108, locking tab 112 is then depressed against housing 108. In response to locking tab 112 being depressed, the prescription bottle and/or lid assembly 106 are rotated relative to each other until locking tab 112 reaches a groove (e.g., groove 314 of FIG. 3) formed by lid assembly 106. In response to the locking tab reaching the groove, locking tab 112 decompresses and remains secured in the groove formed by lid assembly 106. In some embodiments, locking tab 112 may only be removed from the groove formed by lid assembly 106 using a unique tool unavailable to the prescription recipient in order to dissuade the prescription recipient from misusing the prescribed medicine.

In some embodiments, a product orientation structure 120 covers opening 110 ensuring that only one product rests in a dispensing structure opening (e.g., dispensing structure opening 304 of FIG. 3) so that upon reaching opening 110 (see also e.g., opening 312 of FIG. 3) only one product falls through opening 110 at a time. In some embodiments, product orientation structure 120 allows only on product at a time to enter the dispensing structure opening (e.g., dispensing structure opening 304 of FIG. 3) and to be subsequently rotated underneath product orientation structure and over the opening 110.

In some embodiments, a fastener 116 may fasten container 102 to lid assembly 106. In some embodiments, fastener 116 may be a nut and may fasten by screwing on to a threaded structure 118 of the lid assembly 106. In some embodiments, fastener 116 may screw, tighten, lock, clamp, snap, rivet, pin. etc. container 102 to lid assembly 106.

In some embodiments, the lid assembly 106 may further include a sensor disposed in opening 110 and configured to provide sensor data to the processing device 150 indicative that the portion of the products have passed through the opening. In some embodiments, the sensor includes at least one of an infrared sensor, a motion sensor, a magnetic sensor, a proximity sensor, an optical sensor, a photoelectric sensor, a weight sensor, a hall effect sensor, a light sensor, or a mechanical trigger. In some embodiments, the sensor is operatively couple to opening 110 and may sense when a product pass into opening 110.

In some embodiments, the sensor is a photoelectric sensor that provides sensor data used by the processing device 150 to detect pills by difference in color and/or distance. In some embodiments, the sensor is a weight sensor that provides sensor data used by the processing device 150 to detect when a pill places weight on the weight sensor disposed in the opening formed by the hole. In some embodiments, the sensor is a mechanical button or a retractable protrusion disposed in opening 110 formed by the lid assembly 106 that is compressed by the pill traveling through the opening.

In some embodiments, a bottle may have a locking tab that can be depressed and unlocked. The locking lid assembly has grooves (e.g., groove 314 of FIG. 3) that catch the locking tab 112, but the tab cannot be depressed because it is covered by the lid assembly 106. In some embodiments, the locking tab 112 cannot be depressed after locking into a groove inside docking area (i.e., housing of the lid assembly).

In some embodiments, a bottom surface of lid assembly 106 may form an bottom opening 160. In some embodiments, processing device 150 may be disposed within bottom opening 160. In some embodiments, a bottom plate (e.g., bottom plate 318 of FIG. 3) covers bottom opening 160 and may be couple to bottom opening 160 via a shaft (e.g., shaft 316 of FIG. 3). In some embodiments, a receiving aperture 162 may be disposed within bottom opening 160 and may be configured to receive a unique tool to unfasten container 102 from lid assembly 106. In some embodiments, the unique tool may not be able to access the receiving aperture via bottom opening 160 because of obstruction from a bottom plate or base plate (bottom plate 318) covering bottom opening 160. In some embodiments, the unique tool may gain access receiving aperture 162 through a receiving aperture opening 164 disposed on the lid assembly.

In some embodiments, the container 102 may be a medication bottle.

In some embodiments, the dispensing structure 302 may be a rotating dispenser forming an opening (e.g., dispensing opening 304) configured to receive the portion of the products.

In some embodiments, lid assembly 106 may further include a sensor configured to provide sensor data to processing device 150 indicative that the portion of the products have passed through opening 110. In some embodiments, the sensor includes at least one of an infrared sensor, a motion sensor, a magnetic sensor, a proximity sensor, an optical sensor, a photoelectric sensor, a weight sensor, a hall effect sensor, a light sensor, or a mechanical trigger.

In some embodiments, processing device 150 may reset a timer and actuate cover structure (e.g., cover structure 306 of FIG. 3) to cover opening 110, responsive to receiving, from the sensor, sensor data indicative that the portion of the plurality of products have passed through opening 110.

In some embodiments, system 100 further includes a user interface coupled to the processing device, where the user interface is configured to display medication data including at least one of dosage data, dosage interval data, or dosage duration data.

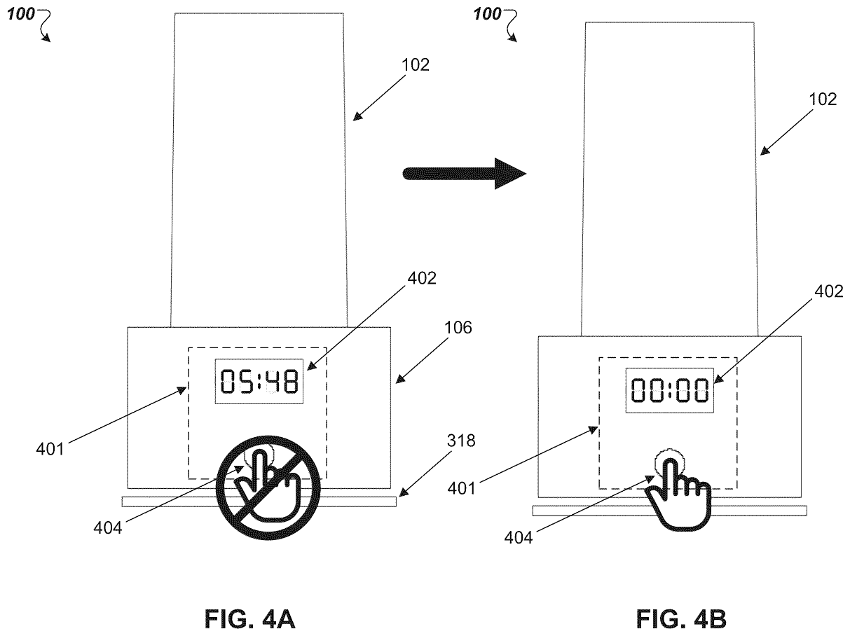

In some embodiments, the system further includes a user interface (e.g., user interface 401 of FIG. 4) coupled to processing device 150, where the user interface is configured to receive user input associated with at least one of a predetermined amount of time or the portion of the products to be dispensed via opening 110. In some embodiments, processing device 150 is configured to determine that the portion of the products is to be dispensed based on the user input.

In some embodiments, system 100 further includes a user interface (e.g., user interface 401 of FIG. 4) coupled to processing device 150, where the user interface is configured to receive user input associated with actuating cover structure (e.g., cover structure 306 of FIG. 3) to uncover opening 110 and to allow the portion of the products to pass through opening 110. In some embodiments, processing device 150 is configured to determine that the portion of the products is to be dispensed based on the user input.

In some embodiments, the lid assembly 106 may include one or more components coupled to (e.g., disposed within, disposed on, etc.) the lid assembly 106. The one or more components may include one or more of a processing device, a user interface, a display screen, a clock, a timer, one or more release mechanisms to release medication (e.g., to release one or more pills from the lid or the bottle for use by the user), a battery, a sensor, wireless module, and/or the like. To conserve power, at least one of the one or more components may be dormant (e.g., turned off) until a predetermined action occurs (e.g., the lid and bottle are secured against each other through use of a button on the lid being pressed by the bottle, actuation of the user interface, and/or the like). The battery of the lid may power at least one of the one or more components.

The processing device may include a timer (e.g., in software or firmware executed by the processing device) or may be coupled to a timer. The timer may be set according to prescription instructions (e.g., administer one pill every 12 hours) and may reset each time a pill is released from the bottle. The user interface (e.g., display screen) may display the amount of time left before the predetermined amount of time of the timer expires in order to inform the prescription recipient of the next available release of a pill.

In some embodiments, the processing device 150 of the lid assembly 106 may (i) be connected to the internet or an emergency network and (ii) track the amount of time that has passed since the predetermined amount of time of the timer had expired. Responsive to the amount of time that has passed meeting a threshold amount, the processing device 150 of the lid may transmit an alert to one or more of the prescription recipient or a third party to either remind the prescription recipient to take the prescribed medicine or to determine whether the prescription recipient is incapacitated and/or needs medical attention.

In some embodiments, the lid assembly 106 may include an internal scale (e.g., sensor). The internal scale may provide sensor data indicative of the overall weight of the pills inside of the bottle for the processing device 150 to determine the number of pills inside of the bottle (e.g., container 102). Processing device 150 may determine how many pills should be remaining in the bottle (e.g., responsive to a predetermined amount of pills being used over a predetermined amount of time). Responsive to the processing device 150 determining there are less pills in the bottle than there should be, the processing device 150 may notify the pharmacist or other third party that the bottle and/or lid might have been tampered with. The processing device 150 may notify the third party of the potential tampering either (i) when the bottle and/or lid is returned to the pharmacist and is connected to a pharmacist's computing device or (ii) over a wireless connection to the internet or the emergency network.

In some embodiments, a movable arm (e.g., a plunger) is used to manipulate a pill from (i.e., out of) the opening 110.

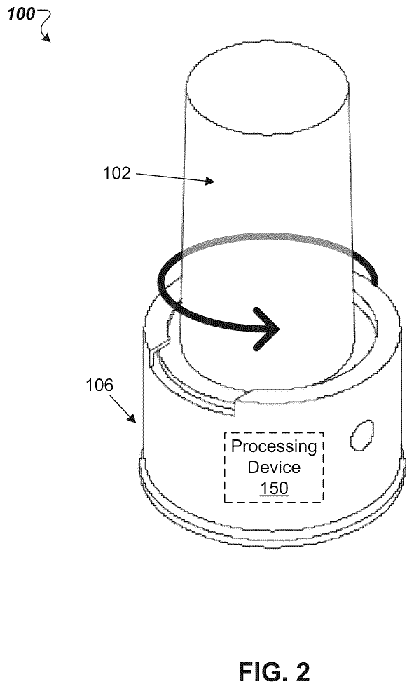

FIG. 2 illustrates a perspective top view of a container locking lid system 100, according to certain embodiments. In some embodiments, one or more components of FIG. 2 have the same or similar functionality, features, material, and/or the like as similarly named and/or numbered components of FIGS. 1A-B.

In some embodiments, in response to a lid assembly 106 being placed on a container 102 (e.g., over the opening formed by container 102), the lid assembly 106 and/or container 102 may swivel opposite each other until the container 102 and lid assembly 106 securely lock to each other. In some embodiments, lid assembly 106 may includes a processing device 150. In some embodiments, processing device 150 is situated inside lid assembly 106.

In some embodiments, once the container 102 and lid assembly 106 are securely locked to each other, the container 102 and lid assembly may not be separated via a conventional tool (e.g., screwdriver, a thin rod to push a button etc.). In some embodiments, the container 102 and lid assembly 106 may only be separated through the use of a unique tool design as to deter the medication recipient from removing the lid assembly 106 to access more pills than prescribed. In some embodiments, a unique tool may be inserted into a receiving aperture (e.g., receiving aperture 162 of FIG. 1) to unfasten container 102 from lid assembly 106. In some embodiments, the receiving aperture may be disposed in the bottom opening (e.g., bottom opening 160 of FIG. 1) of the lid assembly 106. In some embodiments, the lid assembly 106 and container 102 are oriented so that the container 102 is above the lid assembly 106, forcing the pills in the container 102 to rest against the lid assembly 106 via gravity.

FIG. 3 illustrates an exploded perspective top view of the container locking lid system 100, according to certain embodiments. In some embodiments, one or more components of FIG. 3 have the same or similar functionality, features, material, and/or the like as similarly named and/or numbered components of FIGS. 1A-2.

In some embodiments, a dispensing structure 302 may be a rotating dispenser forming a dispenser opening 304 configured to receive the portion of the products. In some embodiments, dispensing structure 302 includes a shaft 316 that is operatively coupled to a bottom plate 318. Bottom plate 318 may be rotated causing shaft 316 to rotate and in turn dispensing structure 302. In some embodiments, such rotation causes a pill to be loaded into the dispensing structure 302 and to be dispensed via opening 312 when uncovered.

In some embodiments, a cover structure 306 is operatively coupled to a rotational arm 308. In some embodiments, rotational arm 308 is operatively coupled to an actuator that when actuated causes cover structure 306 to move. In some embodiments, cover structure 306 is disposed to cover an opening 312 formed by the lid assembly. In some embodiments, cover structure 306 uncovers the opening when actuated to allow contents of the container to pass through.

In some embodiments, a processing device 150 may reset a timer and actuate the cover structure 306 to cover opening 312, responsive to receiving, from the sensor, sensor data indicative that the portion of the products have passed through opening 312.

In some embodiments, the container locking lid system 100 further includes a user interface (e.g., including display 310 and button 311) coupled to the processing device 150, where the user interface is configured to display medication data including at least one of dosage data, dosage interval data, or dosage duration data.

In some embodiments, the container locking lid system 100 further includes a user interface (e.g., including display 310 and button 311) coupled to the processing device 150, where the user interface is configured to receive user input (e.g., a button press of button 311) associated with actuating the cover structure 306 to uncover the opening 312 and to allow the portion of the products to pass through the opening 312, where the processing device 150 is configured to determine that the portion of the products is to be dispensed based on the user input (e.g., button press of button 311).

In some embodiments, the container (e.g., container 102 of FIG. 1) may be a medication bottle.

In some embodiments, the products may be medication in solid dosage form, where the portion of the products corresponds to a dosage of the medication.

In some embodiments, the dispensing structure 302 may be a rotating dispenser forming an opening (e.g., dispenser opening 304) configured to receive the portion of the products.

In some embodiments, the lid assembly 106 may further include a sensor configured to provide sensor data to processing device 150 indicative that the portion of the products have passed through opening 312. In some embodiments, the sensor may include at least one of an infrared sensor, a motion sensor, a magnetic sensor, a proximity sensor, an optical sensor, a photoelectric sensor, a weight sensor, a hall effect sensor, a light sensor, or a mechanical trigger.

In some embodiments, processing device 150 may reset a timer and actuate the cover structure 306 to cover opening 312, responsive to receiving, from the sensor, sensor data indicative that the portion of the products have passed through opening 312.

In some embodiments, the lid assembly may further include a user interface (e.g., display 310 and button 311) coupled to processing device 150. In some embodiments, user interface is configured to display medication data, comprising at least one of dosage data, dosage interval data, or dosage duration data.

In some embodiments, the lid assembly may further include a user interface (e.g., display 310 and button 311) coupled to processing device 150. In some embodiments, user interface (e.g., display 310 and button 311) is configured to receive user input associated with at least one of a predetermined amount of time or the portion of the products to be dispensed via opening 312. In some embodiments, the processing device may be configured to determine that the portion of the products is to be dispensed based on the user input (e.g., via button 311).

FIGS. 4A-B illustrate a user interface of a container locking lid system 100, according to certain embodiments. In some embodiments, one or more components of FIG. 4A-B have the same or similar functionality, features, material, and/or the like as similarly named and/or numbered components of FIGS. 1A-3.

In some embodiments, a timer shown on user interface 401 (e.g., including display 402 and button 404) tracks a remaining amount of time before a dosage may be dispensed. When the timer reaches zero, button 404 may be pressed by a user and the cover structure will uncover the opening (e.g., opening 312 of FIG. 3). User may then rotate bottom plate 318, causing the shaft (e.g., shaft 316 of FIG. 3) and dispensing structure (e.g., dispensing structure 302 of FIG. 3) to rotate and load a pill into the opening (e.g., dispenser opening 312 of FIG. 3). In response to the sensor sending sensor data to processing device 150 indicating that the pill has passed through the opening, the processing device 150 causes cover structure (e.g., cover structure 306 of FIG. 3) to be actuated to cover the opening. In response to the sensor sending sensor data to the processing device 150 indicating that the pill has passed through the opening, the processing device 150 also resets the timer. In some embodiments, the timer is displayed on display 402.

In some embodiments, the lid may include a user interface (e.g., an indicator) that displays a message to one or more of the prescription recipients or the pharmacist as to how well the patient is adhering to the medication schedule. The user interface (e.g., indicator) may include a light emitting diode (LED) that changes color (e.g., from green to red) (i) if the bottle and/or lid have undergone tampering, (ii) if the prescription recipient is late taking the medication, and/or the like. The user interface (e.g., indicator) may include a display screen that provides details of how well the patient is adhering to the medication schedule (e.g., a sliding scale from 1-10, displaying messages as to how the prescription recipient may improve their medication consumption, and/or the like).

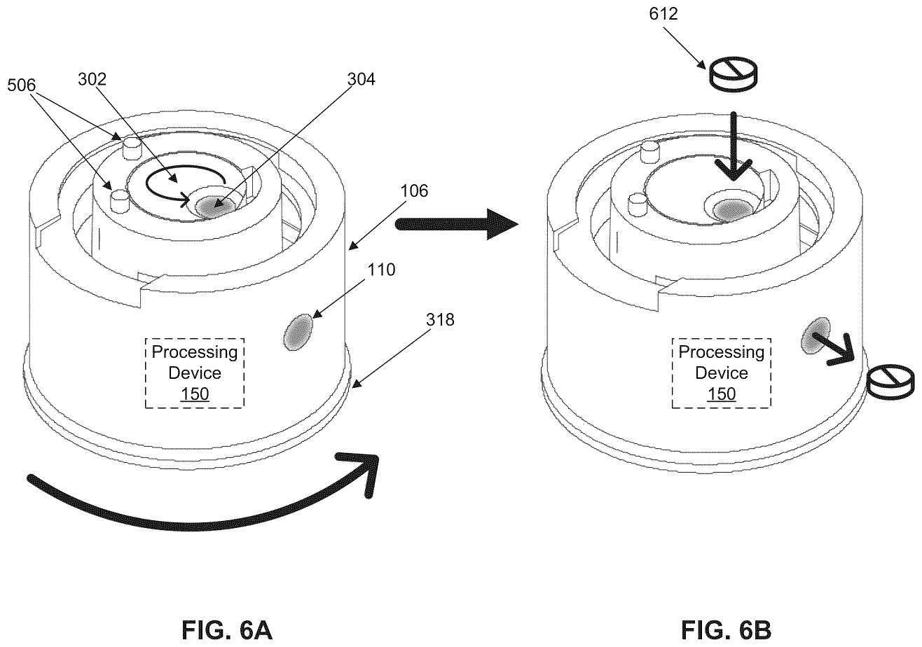

In some embodiments, if user interface button 404 is pressed before the timer has reached zero, the cover structure (e.g., cover structure 306 of FIG. 3 will not actuate and the opening (e.g., opening 110) will remain covered. A user may rotate bottom plate 318 freely but no product (e.g., pill 612 of FIG. 6) will be dispensed because the opening remains covered. In this way products are safely contained inside the container 102 until the next dose is ready (i.e., timer finishes counting down).

In some embodiments, processing device 150 will actuate a cover structure (e.g., cover structure 306 of FIG. 3) in response to the timer reaching zero (in such an embodiment no user input to button 404 is needed).

In some embodiments, display 402 may be an LCD display, LED display, OLED display, e-paper display, CRT display, plasma display, projection display, touchscreen display, segmented display, dot-matrix display, VFD (vacuum fluorescent display), RGB LED display, backlit display, monochrome display, color display, electroluminescent display, flip-dot display, seven-segment display, alphanumeric display, graphic display, holographic display, heads-up display, flashing light display (such as a red, yellow, or green light) and/or the like.

In some embodiments, button 404 may be a push button, toggle button, rocker switch, slide switch, rotary encoder, membrane button, capacitive touch button, tactile switch, soft button, trigger button, arcade button, joystick button, microswitch, momentary button, latching button, reset button, power button, function button, mode button, navigation button, arrow button, dial button, scroll wheel button, trackball button, touchpad button, pressure-sensitive button and/or the like.

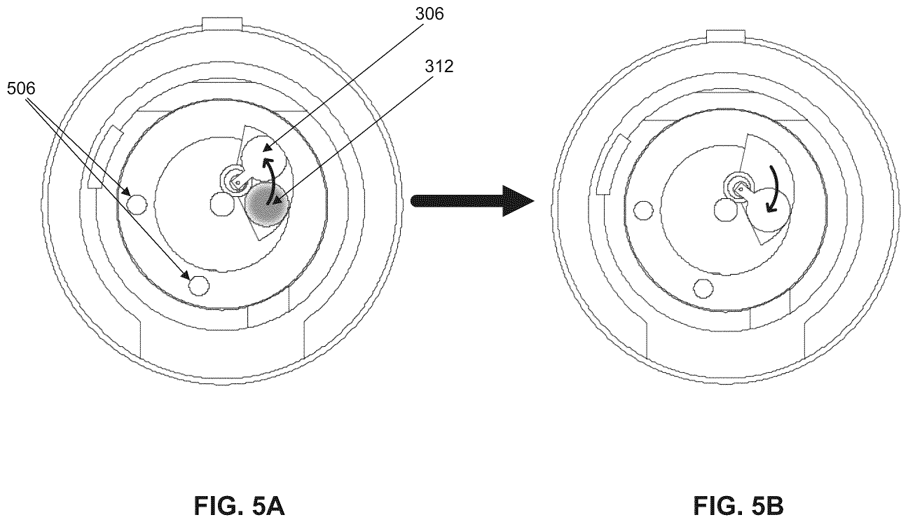

FIGS. 5A-B illustrate a cover structure 306 associated with a container locking lid, according to certain embodiments. In some embodiments, one or more components of FIGS. 5A-B have the same or similar functionality, features, material, and/or the like as similarly named and or numbered components of FIGS. 1A-4B.

In some embodiments, cover structure 306 may be actuated by a processing device (e.g., processing device 150 of FIG. 1) to uncover opening 312. In some embodiments, in response to a sensor sending sensor data to the processing device indicating that a product has entered opening 312, cover structure 306 may be actuated to re-cover (i.e., cover again) opening 312 so that no more product may pass into opening 312.

In some embodiments a user interface (e.g., user interface 401 of FIG. 4) associated with a prescription bottle locking lid includes programming buttons 506. In some embodiments, programming buttons may be used by a pharmacist to program the dosage amount and dosage interval of a prescription container in container 102 of FIG. 1. In some embodiments, the user interface is displayed via a computing device (e.g., smart phone, tablet, computer, etc.). In some embodiments, the user interface is displayed via the prescription bottle locking lid (e.g., on a display screen). The user interface may allow the pharmacist, medical provider, and/or caretaker to determine (i) whether the prescription bottle locking lid is connected to the internet, (ii) whether the prescription recipient has been releasing pills at the regular intervals (e.g., predetermined amounts of time) indicated by prescription instructions, (iii) whether the prescription bottle locking lid has undergone tampering, and/or (iv) information regarding the identity of the prescription recipient and/or information about the prescription (e.g., medication type, dosage level, and/or the like). In some embodiments, the programming interface (e.g., programming buttons 506) may allow the pharmacist to program the prescription bottle locking lid in response to the prescription recipient filling the prescription order. In some embodiments, programming the prescription bottle locking lid includes setting dosage amount, a dosage interval, etc.

In some embodiments, for example, a programming buttons 506 may include a first and a second button. The first button may be pressed, for example, two times to indicated that the dosage is two pills. The second button may be pressed, for example, eight times to indicate that the dosage frequency is every eight hours. In some embodiments, the user interface (e.g., user interface 401 of FIG. 4) may be configured in a variety of ways to facilitate use of a container locking lid system (e.g., system 100 of FIG. 1).

In some embodiments, programming buttons 506 may be a push button, toggle button, rocker switch, slide switch, rotary encoder, membrane button, capacitive touch button, tactile switch, soft button, trigger button, arcade button, joystick button, microswitch, momentary button, latching button, reset button, power button, function button, mode button, navigation button, arrow button, dial button, scroll wheel button, trackball button, touchpad button, pressure-sensitive button and/or the like.

FIGS. 6A-B illustrate container locking lid systems, according to certain embodiments. In some embodiments, one or more components of FIGS. 6A-B have the same or similar functionality, features, material, and/or the like as similarly named and/or numbered components of FIGS. 1A-5B.

In some embodiments, the lid assembly 106 may include one or more programming buttons 506 on the top of the lid (e.g., the portion of the lid inaccessible to the prescription recipient when the lid and the bottle are secured against each other, a portion of the lid that is in the interior volume formed by the lid and bottle) that allow the pharmacist to program the specific prescription instructions (e.g., a predetermined amount of time of the timer, the number of pills per dose, the total number of pills in the bottle or the like) into the processing device 150 of the lid. In some embodiments, programming buttons 506 receive user input. In some embodiments, processing device 150 receives user input and sets the timer, dosage amount, dosages frequency, etc. accordingly.

In some embodiments, a total number of pills may be displayed on the user interface (e.g., display screen) of the lid which is updated (e.g., reset) after each predetermined amount of time of the timer expires, medication is removed from the bottle (e.g., by rotating bottom plate 318, causing the shaft and dispensing structure 302 to rotate and load a pill 612 into the dispensing structure opening 304 and then the lid assembly opening 110). In some embodiments, the products (e.g., pill 612) may be medication in solid dosage form, where the portion of the products corresponds to a dosage of the medication.

In some embodiments, one or more components of the lid (e.g., the processing device 150, the timer, the number of pills per dose, and/or the like) may be programmable by a pharmacist. The one or more components of the lid may be programmed by a pharmacist inputting prescription details into a computing device (e.g., executing a computer application) while the processing device 150 of the lid is connected (via WiFi®, Bluetooth®, wired connection, or the like) to the computing device.

In some embodiments, for example, a programming buttons 506 may include a first and a second button. The first button may be pressed, for example, two times to indicated that the dosage is two pills. The second button may be pressed, for example, eight times to indicate that the dosage frequency is every eight hours. In some embodiments, the user interface (e.g., user interface 401 of FIG. 4) may be configured in a variety of ways to facilitate use of a container locking lid system (e.g., system 100 of FIG. 1).

In some embodiments, when cover structure (e.g., cover structure 306 of FIG. 3) is actuated to uncover opening 110, a user may rotate bottom plate 318 causing dispensing structure 302 to rotate. With bottom plate 318 oriented downward, a product that is contained in a container (e.g., container 102 of FIG. 1) that is coupled to lid assembly 106 may align with dispenser opening 304 and load into dispenser opening 304. In response to the dispensing structure being rotated, the loaded product may be positioned in alignment with opening 110 and may pass through the opening as illustrated in FIG. 6B.

In some embodiments, if user interface button (e.g., button 404 of FIG. 4) is pressed before the timer has reached zero, the cover structure (e.g., cover structure 306 of FIG. 3 will not actuate and opening 110 will remain covered. A user may rotate bottom plate 318 freely but no product (e.g., pill 612) will be dispensed because opening 110 remains covered. In this way products are safely contained inside the container (e.g., container 102 of FIG. 1) until the next dose is ready (i.e., timer finishes counting down).

In some embodiments, processing device 150 will actuate a cover structure (e.g., cover structure 306 of FIG. 3) in response to the timer reaching zero (in such an embodiment no user input to e.g., button 404 of FIG. is needed).

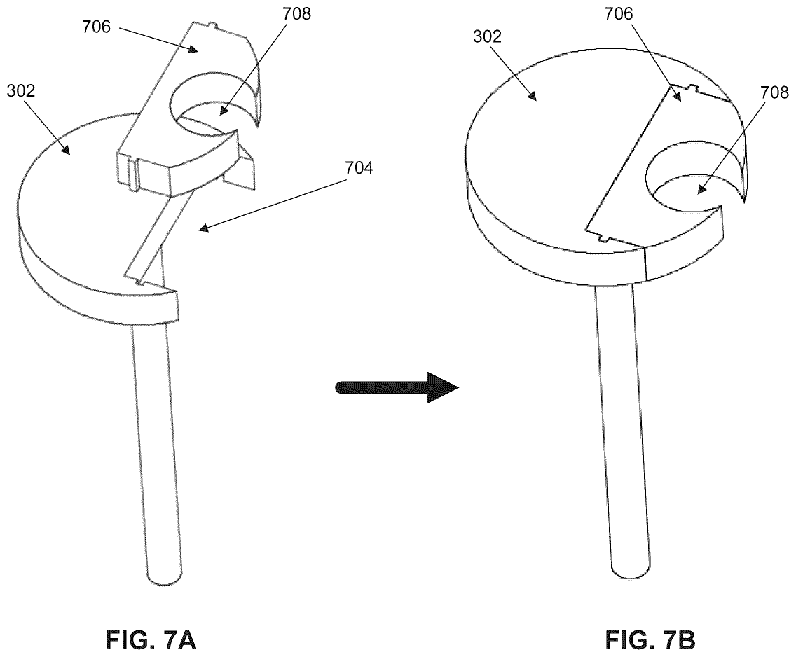

FIGS. 7A-B illustrate a dispenser (e.g., a substantially cylindrical dispenser) of a container locking lid system, according to certain embodiments. In some embodiments, one or more features of FIG. 7A-B have the same or similar functionality, features, material, and/or the like as similarly named and/or numbered components of FIGS. 1A-6B. In some embodiments, removable insert 706 is interchangeable, swappable, exchangeable, modifiable and/or modular. IN some embodiments, removable insert may snap-onto the geometry of the dispensing structure or dispensing structure port 704.

In some embodiments, the locking lid assembly of a container locking lid system may further include a removable insert 706, where the removable insert 706 is removably coupled to a dispensing structure port 704, the removable insert 706 being configured to change the size, shape, or function of the dispensing structure opening 708 when inserted to accommodate different sizes and shapes of medication in solid dosage form. FIG. 7B shows removable insert 706 coupled to dispensing structure 302. In some embodiments, dispensing structure opening 708 may be any shape or size to accommodate different products contained in a container (e.g., container 102 of FIG. 1).

In some embodiments, the products may be medication in solid dosage form. In some embodiments, the portion of the products corresponds to a dosage of the medication.

In some embodiments, the locking lid assembly 106 may further include a removable insert 706, where removable insert 706 is removably coupled to a dispensing structure port 704, removable insert 706 being configured to change the size, shape, or function of dispensing structure opening 708 when inserted to accommodate different sizes and shapes of medication in solid dosage form.

In some embodiments, the dispensing structure may have fan-like shape or any other shape to facilitate dispensing of products.

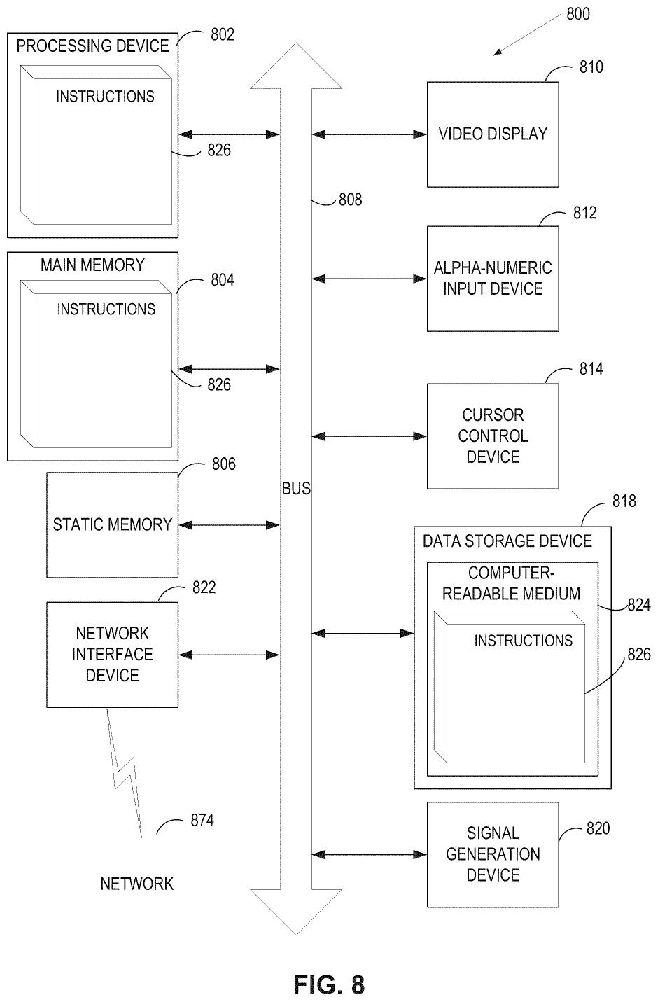

FIG. 8 is a block diagram illustrating a computer system 800, according to certain embodiments. In some embodiments, the computer system 800 is a processing device (e.g., processing device 150 of FIGS. 1-3 and 6) of a container locking lid system.

In some embodiments, computer system 800 is connected (e.g., via a network, such as a Local Area Network (LAN), an intranet, an extranet, or the Internet) to other computer systems. In some embodiments, computer system 800 operates in the capacity of a server or a client computer in a client-server environment, or as a peer computer in a peer-to-peer or distributed network environment. In some embodiments, computer system 800 is provided by a personal computer (PC), a tablet PC, a Set-Top Box (STB), a Personal Digital Assistant (PDA), a cellular telephone, a web appliance, a server, a network router, switch or bridge, or any device capable of executing a set of instructions (sequential or otherwise) that specify actions to be taken by that device. Further, the term “computer” shall include any collection of computers that individually or jointly execute a set (or multiple sets) of instructions to perform any one or more of the methods described herein.

In a further aspect, the computer system 800 includes a processing device 802, a volatile memory 804 (e.g., Random Access Memory (RAM)), a non-volatile memory 806 (e.g., Read-Only Memory (ROM) or Electrically-Erasable Programmable ROM (EEPROM)), and a data storage device 818, which communicate with each other via a bus 808.

In some embodiments, processing device 802 is provided by one or more processors such as a general purpose processor (such as, for example, a Complex Instruction Set Computing (CISC) microprocessor, a Reduced Instruction Set Computing (RISC) microprocessor, a Very Long Instruction Word (VLIW) microprocessor, a microprocessor implementing other types of instruction sets, or a microprocessor implementing a combination of types of instruction sets) or a specialized processor (such as, for example, an Application Specific Integrated Circuit (ASIC), a Field Programmable Gate Array (FPGA), a Digital Signal Processor (DSP), or a network processor).

In some embodiments, computer system 800 further includes a network interface device 822 (e.g., coupled to network 874). In some embodiments, computer system 800 also includes a video display unit 810 (e.g., a liquid-crystal display (LCD)), an alphanumeric input device 812 (e.g., a keyboard), a cursor control device 814 (e.g., a mouse), and a signal generation device 820.

In some implementations, data storage device 818 includes a non-transitory computer-readable storage medium 824 on which store instructions 826 encoding any one or more of the methods or functions described herein.

In some embodiments, instructions 826 also reside, completely or partially, within volatile memory 804 and/or within processing device 802 during execution thereof by computer system 800, hence, in some embodiments, volatile memory 804 and processing device 802 also constitute machine-readable storage media.

While computer-readable storage medium 824 is shown in the illustrative examples as a single medium, the term “computer-readable storage medium” shall include a single medium or multiple media (e.g., a centralized or distributed database, and/or associated caches and servers) that store the one or more sets of executable instructions. The term “computer-readable storage medium” shall also include any tangible medium that is capable of storing or encoding a set of instructions for execution by a computer that cause the computer to perform any one or more of the methods described herein. The term “computer-readable storage medium” shall include, but not be limited to, solid-state memories, optical media, and magnetic media.

The methods, components, and features described herein can be implemented by discrete hardware components or can be integrated in the functionality of other hardware components such as application-specific integrated circuits (ASICS), FPGAs, DSPs or similar devices. In addition, the methods, components, and features can be implemented by firmware modules or functional circuitry within hardware devices. Further, the methods, components, and features can be implemented in any combination of hardware devices and computer program components, or in computer programs.

The above description is intended to be illustrative, and not restrictive. Although the present disclosure has been described with references to specific illustrative examples and implementations, it will be recognized that the present disclosure is not limited to the examples and implementations described. The scope of the disclosure should be determined with reference to the following claims, along with the full scope of equivalents to which the claims are entitled.

The preceding description sets forth numerous specific details such as examples of specific systems, components, methods, and so forth in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular implementations may vary from these exemplary details and still be contemplated to be within the scope of the present disclosure.

The terms “over,” “under,” “between,” “disposed on,” and “on” as used herein refer to a relative position of one material layer or component with respect to other layers or components. For example, one layer disposed on, over, or under another layer may be directly in contact with the other layer or may have one or more intervening layers. Moreover, one layer disposed between two layers may be directly in contact with the two layers or may have one or more intervening layers. Similarly, unless explicitly stated otherwise, one feature disposed between two features may be in direct contact with the adjacent features or may have one or more intervening layers.

The words “example” or “exemplary” are used herein to mean serving as an example, instance or illustration. Any aspect or design described herein as “example” or “exemplary” is not necessarily to be construed as preferred or advantageous over other aspects or designs. Rather, use of the words “example” or “exemplary” is intended to present concepts in a concrete fashion.

Reference throughout this specification to “one embodiment,” “an embodiment,” or “some embodiments” means that a particular feature, structure, or characteristic described in connection with the embodiment is included in at least one embodiment. Thus, the appearances of the phrase “in one embodiment,” “in an embodiment,” or “in some embodiments” in various places throughout this specification are not necessarily all referring to the same embodiment. In addition, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Also, the terms “first,” “second,” “third,” “fourth,” etc. as used herein are meant as labels to distinguish among different elements and can not necessarily have an ordinal meaning according to their numerical designation. When the term “about,” “substantially,” or “approximately” is used herein, this is intended to mean that the nominal value presented is precise within ±10%.

Although the operations of the methods herein are shown and described in a particular order, the order of operations of each method may be altered so that certain operations may be performed in an inverse order so that certain operations may be performed, at least in part, concurrently with other operations. In another embodiment, instructions or sub-operations of distinct operations may be in an intermittent and/or alternating manner.

It is understood that the above description is intended to be illustrative, and not restrictive. Many other embodiments will be apparent to those of skill in the art upon reading and understanding the above description. The scope of the disclosure should, therefore, be determined with reference to the appended claims, along with the full scope of equivalents to which such claims are entitled.

What is claimed is:

1. A system comprising:
a container forming an interior volume configured to store a plurality of products; and
a lid assembly configured to secure to the container, the lid assembly comprising:
a lid housing forming an opening, wherein the plurality of products are to be dispensed from the interior volume via the opening;
a dispensing structure configured to load a portion of the plurality of products to be dispensed;
a cover structure configured to selectively unblock the opening to allow the portion of the plurality of products to be dispensed;
a processing device configured to:
determine that the portion of the plurality of products is to be dispensed; and
cause the cover structure to be actuated to unblock the opening to allow dispensing of the portion of the plurality of products.

2. The system of claim 1, wherein the container is a medication bottle.

3. The system of claim 1, wherein the plurality of products are medication in solid dosage form, wherein the portion of the plurality of products corresponds to a dosage of the medication.

4. The system of claim 1, wherein the dispensing structure is a rotating dispenser forming an opening configured to receive the portion of the plurality of products.

5. The system of claim 1, wherein the lid assembly further comprises a sensor configured to provide sensor data to the processing device indicative that the portion of the plurality of products have passed through the opening, wherein the sensor comprises at least one of an infrared sensor, a motion sensor, a magnetic sensor, a proximity sensor, an optical sensor, a photoelectric sensor, a weight sensor, a hall effect sensor, a light sensor, or a mechanical trigger.

6. The system of claim 5, wherein the processing device is to reset a timer and actuate the cover structure to cover the opening, responsive to receiving, from the sensor, sensor data indicative that the portion of the plurality of products have passed through the opening.

7. The system of claim 1 further comprising a user interface coupled to the processing device, wherein the user interface is configured to display medication data comprising at least one of dosage data, dosage interval data, or dosage duration data.

8. The system of claim 1 further comprising a user interface coupled to the processing device, wherein the user interface is configured to receive user input associated with at least one of a predetermined amount of time or the portion of the plurality of products to be dispensed via the opening, wherein the processing device is configured to determine that the portion of the plurality of products is to be dispensed based on the user input.

9. The system of claim 1 further comprising a user interface coupled to the processing device, wherein the user interface is configured to receive user input associated with actuating the cover structure to uncover the opening and to allow the portion of the plurality of products to pass through the opening, wherein the processing device is configured to determine that the portion of the plurality of products is to be dispensed based on the user input.

10. A locking lid assembly comprising:
a lid housing forming an opening, wherein the housing is configured to secure a container, the container forming an interior volume configured to store a plurality of products, and wherein the plurality of products are to be dispensed from the interior volume via the opening;
a dispensing structure configured to load a portion of the plurality of products to be dispensed;
a cover structure configured to selectively unblock the opening to allow the portion of the plurality of products to be dispensed;
a processing device configured to:
determine that the portion of the plurality of products is to be dispensed; and
cause the cover structure to be actuated to unblock the opening to allow dispensing of the portion of the plurality of products.

11. The locking lid of claim 10, wherein the container is a medication bottle.

12. The locking lid of claim 10, wherein the plurality of products are medication in solid dosage form, wherein the portion of the plurality of products corresponds to a dosage of the medication.

13. The locking lid of claim 10, wherein the dispensing structure is a rotating dispenser forming an opening configured to receive the portion of the plurality of products.

14. The locking lid of claim 10, wherein the lid assembly further comprises a sensor configured to provide sensor data to the processing device indicative that the portion of the plurality of products have passed through the opening, wherein the sensor comprises at least one of an infrared sensor, a motion sensor, a magnetic sensor, a proximity sensor, an optical sensor, a photoelectric sensor, a weight sensor, a hall effect sensor, a light sensor, or a mechanical trigger.

15. The locking lid of claim 14, wherein the processing device is to reset a timer and actuate the cover structure to cover the opening, responsive to receiving, from the sensor, sensor data indicative that the portion of the plurality of products have passed through the opening.

16. The locking lid of claim 10 further comprising a user interface coupled to the processing device, wherein the user interface is configured to display medication data, comprising at least one of dosage data, dosage interval data, or dosage duration data.

17. The locking lid of claim 10 further comprising a user interface coupled to the processing device, wherein the user interface is configured to receive user input associated with at least one of a predetermined amount of time or the portion of the plurality of products to be dispensed via the opening, wherein the processing device is configured to determine that the portion of the plurality of products is to be dispensed based on the user input.

18. A locking lid assembly comprising:
a lid housing forming an interior volume, the lid housing further forming a lid housing opening coupled to the interior volume; and
a substantially cylindrical dispensing structure disposed in the interior volume of the lid housing, the substantially cylindrical dispensing structure forming a dispensing structure opening configured to receive one or more products, wherein the substantially cylindrical dispensing structure is configured to rotate to cause the one or more products disposed in the dispensing structure opening to be dispensed through the lid housing opening.

19. The locking lid assembly of claim 18, wherein the one or more products are medication in solid dosage form, wherein a portion of the one or more products corresponds to a dosage of the medication.

20. The locking lid assembly of claim 19, further comprising a removable insert, wherein the removable insert is removably coupled to a dispensing structure port, the removable insert being configured to change a size, shape, or function of the dispensing structure opening when inserted to accommodate different sizes and shapes of medication in solid dosage form.
